(19) United States
(12) Patent Application Publication
(10) Pub. No.: US 2025/0222246 A1
(43) Pub. Date: Jul. 10, 2025

(54) NEEDLE FREE CONNECTOR WITH ANTI-MICROBIAL PROPERTIES

(71) Applicant: CareFusion 303, Inc., San Diego, CA (US)

(72) Inventors: Ryan Callahan, Long Beach, CA (US); Siddarth K. Shevgoor, Mission Viejo, CA (US)

(21) Appl. No.: 18/404,261

(22) Filed: Jan. 4, 2024

(51) Int. Cl. A61M 39/16; A61M 39/22

(52) U.S. Cl. CPC A61M 39/16; A61M 39/22

(57) ABSTRACT

A needle free connector including a first component and a second component is disclosed herein. The first component includes a housing having a proximal end and a distal end opposite the proximal end, the housing defines an interior volume. A center post is disposed in the interior volume extending proximally in the interior volume, a valve is disposed in the interior volume at the proximal end of the first component surrounding at least a portion of the center post; and a lubricant is disposed between the center post and the valve. The lubricant comprises an antimicrobial component. The second component includes a casing having a proximal end and a distal end opposite the proximal end, the casing defines a fluid path therein. A plug is disposed in the fluid path at the distal end of the second component.

### TECHNICAL FIELD

The present disclosure generally relates to needle free connectors and, in some embodiments, to a needle free connector with anti-microbial properties.

### BACKGROUND

Force-activated separation devices (FASD) in intravenous (IV) sets are vital safety features designed to prevent accidental disconnections. These devices are engineered to disconnect the IV line when a certain force is applied, ensuring the secure delivery of fluids to patients. This breakaway is intended to prevent the damage or dislodgement of components of the infusion system that are more critical, such as a catheter. They are especially useful in clinical settings to mitigate the risk of unintentional line dislodgement, which could lead to complications or infections. FASDs in IV sets enhance patient safety by maintaining the integrity of the infusion system.

### SUMMARY

There are two main categories of FASD, single use and reconnectable. Single use devices are replaced in the event of a breakaway. Re-usable devices can be reconnected after a separation of the components. Needle free connectors, and example of re-usable devices, are designed to maintain closed access to an infusion system when not in use and can allow for repeated access by luer devices (e.g., syringes, IV sets, etc.). As the mating surfaces of the devices can become contaminated when disconnected there is a risk of central line-associated bloodstream infection (CLABSI) or other infections when reconnecting a contaminated device. The clinician must be sure to scrub each surface with disinfectant per the device manufacturers guidance. If a patient or patient family member were to reconnect without disinfection, there is a high likelihood of an infection developing.

As such, there is a need for adding anti-microbial properties to the FASDs to allow or greatly reduce the risk of injection when connected without any disinfection procedure. The valve side of the FASD often contains silicone lubricant between the valve and the center post to assist in smooth motion of the plug side plug sliding over the valve. During connection, this lubricant coats the face of the valve side valve and the interior of the plug side plug. By adding an antimicrobial compound into the silicone lubricant mixture at a specified concentration, bacteria that contacts the lubricated surfaces will be killed. The location of lubrication being in the core fluid path of the device and covering most surfaces after connection, along with the bacterial killing properties of the antimicrobial compound impregnated lubricant results in a connector that may not need any disinfection step while still ensuring a microbe free fluid path.

In certain embodiments, a needle free connector comprises a first component comprising a housing having a proximal end and a distal end opposite the proximal end, the housing defining an interior volume; a center post disposed in the interior volume extending proximally in the interior volume; a valve disposed in the interior volume at the proximal end of the first component surrounding at least a portion of the center post; and a lubricant disposed between the center post and the valve. In certain embodiments, the lubricant comprises an antimicrobial component.

In certain embodiments, a second component comprises a casing having a proximal end and a distal end opposite the proximal end, the casing defining a fluid path therein; and a plug disposed in the fluid path at the distal end of the second component. In certain embodiments, the plug is a deformable member. In certain embodiments, the plug comprises a septum extending therethrough. In certain embodiments, the septum is sized and shaped to receive a portion of the center post. In certain embodiments, the septum is coated with the lubricant when the center post is disposed therethrough.

In certain embodiments, the proximal end of the first component and the distal end of the second component are spaced apart in a disconnected configuration. In certain embodiments, the proximal end of the first component and the distal end of the second component are removably coupled in a connected configuration. In certain embodiments, the first component and the second component are in fluid communication in the connected configuration. In certain embodiments, the valve deforms longitudinally in the distal direction to expose the center post in the connected configuration.

In certain embodiments, the center post is a rigid member. In certain embodiments, the antimicrobial component comprises chlorohexidine gluconate. In certain embodiments, a concentration of the antimicrobial component is between 4%-6% wt/wt of the lubricant.

In certain embodiments, a method of reducing a presence of microbes in a needle free connector comprises providing a first component comprising a valve and a center post; providing a second component comprising a plug; providing a lubricant to an area between the center post and the valve; connecting the first component and the second component such that the plug displaces the valve and surrounds the center post, wherein the lubricant is transferred to the plug when the first component is connected to the second component, and wherein the lubricant comprises an antimicrobial component.

In certain embodiments, the first component further comprises: a housing having a proximal end and a distal end opposite the proximal end, the housing defining an interior volume; the center post disposed in the interior volume extending proximally in the interior volume; and the valve disposed in the interior volume at the proximal end of the first component surrounding at least a portion of the center post.

In certain embodiments, the second component further comprises: a casing having a proximal end and a distal end opposite the proximal end, the casing defining a fluid path therein; and the plug disposed in the fluid path at the distal end of the second component.

In certain embodiments, the plug comprises a septum extending therethrough. In certain embodiments, the septum is sized and shaped to receive a portion of the center post. In certain embodiments, the septum is coated with the lubricant when the center post is disposed therethrough. In certain embodiments, the antimicrobial component comprises chlorohexidine gluconate. In certain embodiments, the concentration of the antimicrobial component is between 4%-6% wt/wt of the lubricant.

### BRIEF DESCRIPTION OF THE DRAWINGS

The foregoing summary, as well as the following detailed description of embodiments of the assembly, will be better understood when read in conjunction with the appended drawings of exemplary embodiments. It should be understood, however, that the invention is not limited to the precise arrangements and instrumentalities shown.

In the drawings:

FIG. 1 is a schematic view an IV set coupled to a patient in accordance with exemplary embodiments of the present disclosure;

FIG. 2 is a top perspective view of a needle free connector assembly in accordance with exemplary embodiments of the present disclosure;

FIG. 3 is a side cross-sectional view of a needle free connector in a disconnected configuration in accordance with exemplary embodiments of the present disclosure;

FIG. 4 is a side cross-sectional view of a needle free connector in a connected configuration in accordance with exemplary embodiments of the present disclosure; and

FIG. 5 is a side cross-sectional view of a valve side of a needle free connector in accordance with exemplary embodiments of the present disclosure.

### DETAILED DESCRIPTION

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to reducing the presence of microbes by utilizing the disclosed needle free connector with anti-microbial properties, it is to be understood that this description is only an example of usage and does not limit the scope of the claims. Various aspects of the disclosed needle free connector may be used in any application where it is desirable to reduce the presence of microbes in a needle free connector.

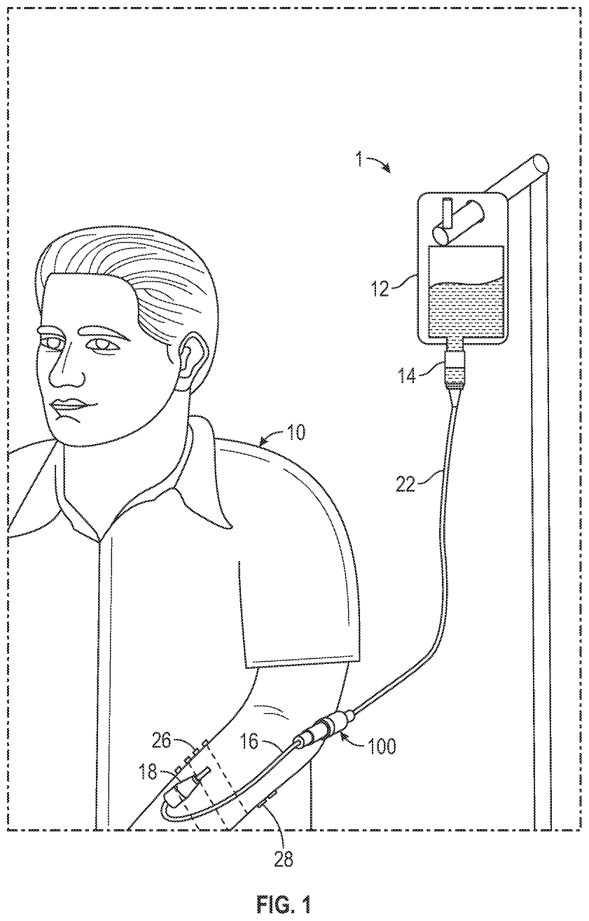

Referring now to the figures, FIG. 1 illustrates an IV set 1 coupled to a patient 10, in accordance with aspects of the present disclosure. The IV set 1 includes a medicament bag 12, a drip chamber 14, and tubing 22. The tubing 22 extends between the drip chamber 14 and a fluid connector assembly 100 of the IV set 1. To resist unintended dislodgement or disconnection of the tubing 16 or the catheter 18 from the patient, tape 26 is placed over the tubing 16 and the catheter 18, so that the tape 26 engages the tubing 16, the catheter 18, and the patient 10.

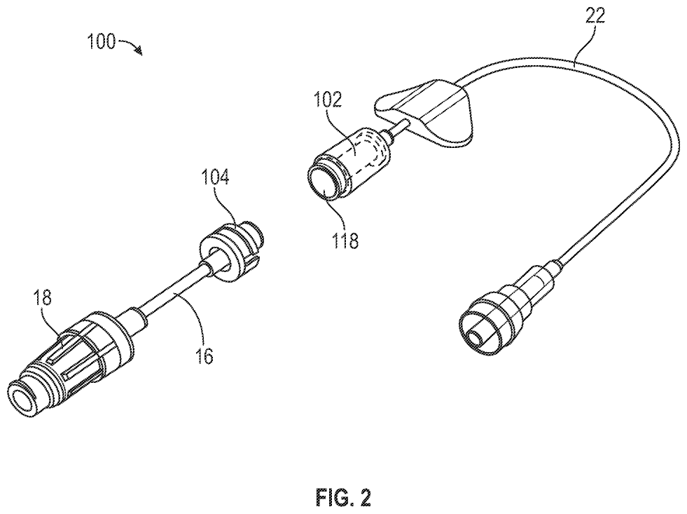

FIG. 2 illustrates a perspective view of a fluid connector assembly 100 for use with an IV set, in accordance with aspects of the present disclosure. The fluid connector assembly 100 is designed for use in medical applications, such as the IV set 1 (shown in FIG. 1) as well as other medical fluid delivery applications using catheters, including peripheral intravenous catheters (“PIVC”), as non-limiting examples.

As shown in FIGS. 2-5, the fluid connector assembly 100 may include a first component 102 and a second component 104. The first component 102 may be coupled to tubing 22 or tubing 16. In some embodiments, the first component 102 is closer to the medicament bag 12 than the second component 104. In some embodiments, the second component 104 is closer to the medicament bag 12 than the first component 102.

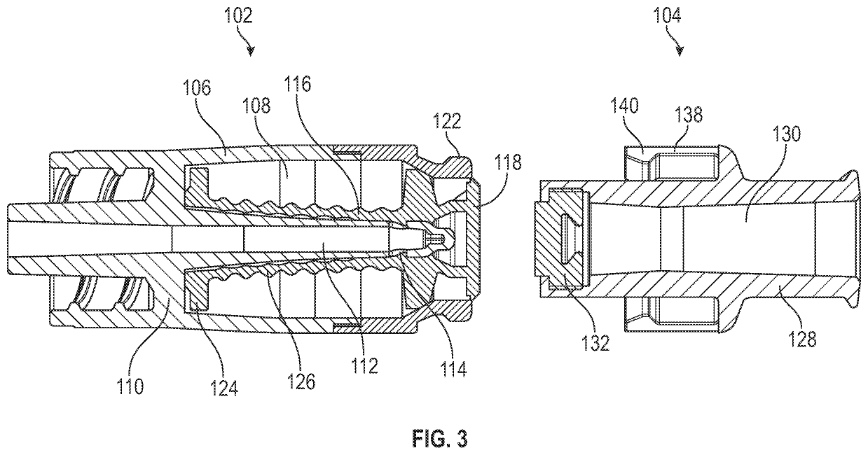

As shown in FIG. 3, the first component may include a housing 106 having a proximal end and a distal end opposite the proximal end. As shown in FIG. 3, the proximal end may be to the right of the distal end. It would be appreciated that in some embodiments, the proximal end may be to the left of the distal end. The housing 106 may define an interior volume 108. The interior volume 108 may be generally cylindrical and may include components disposed therein. The interior volume 108 may extend from the proximal end toward the distal end and may include a wall 110 on the distal most portion. The housing 106 may have a collar 122 at the proximal end thereof. The collar 122 may extending radially outward around the circumference of the proximal end.

As shown in FIGS. 3-5, the interior volume 108 may include a center post 112 disposed therein. The center post 112 may be a rigid member and may extend proximally in the interior volume 108. The center post 112 may be coupled to the housing 106 and may extend through the wall 110. In some embodiments, the center post 112 is integrally formed with the wall 110. In some embodiments, the housing 106, the center post 112 and the wall 110 may be integrally formed. The center post 112 may be a generally conical shaped member. A proximal end of the center post 112 may have a smaller circumference than the distal end of the center post 112. In some embodiments, the center post 112 may be cylindrical. The center post 112 includes a fluid inlet 114 extending therethrough to receive a fluid medicament, as described in more detail below. In some embodiments, the center post 112 includes a plurality of fluid inlets 114. The fluid inlet 114 may allow the fluid medicament to travel from the proximal end of the first component 102 to the distal end and into tubing 16. In some embodiments, the fluid inlet 114 may allow the fluid medicament to travel from the distal end to the proximal end of the first component 102 and into the second component 104.

As shown in FIGS. 3-5, the interior volume 108 may include a valve 116 disposed therein. The valve 116 may be located at the proximal end of the first component 102 and may extend distally to surround at least a portion of the center post 112. The valve 116 may be a flexible member. The valve 116 may be made of a rubber, silicone, or other deformable material. The valve 116 may have a generally cylindrical base 118. The base 118 may include a slit 120, as shown in FIG. 5, extending therethrough. The slit 120 may be shaped and sized to expose the fluid inlet 114 of the center post 112 in response to a force deforming the valve 116 distally. The valve 116 may have a flare 124 at a distal end thereof. The flare 124 may engage the wall 110 to prevent the distal end of the valve 116 from moving distally in response to a force deforming the valve 116 distally. The valve 116 may have a body 126 extending between the base 118 and the flare 124. The body 126 may be in contact with the center post 112. The body 126 may be ribbed to promote folding over itself in response to a force deforming the valve 116 distally.

The first component 102 may include a lubricant 136 disposed between the center post 112 and the valve 116. The lubricant 136 may include an antimicrobial component. The antimicrobial component may be chlorohexidine gluconate. The antimicrobial component may have a concentration between 4%-6% wt/wt of the lubricant 136. In some embodiments, the antimicrobial component may be at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, or at least 8%, of the lubricant 136. In some embodiments, the antimicrobial component may have a concentration between 1%-8% wt/wt, between 1.5%-7.5% wt/wt, between 2%-7% wt/wt, between 2.5%-6.5% wt/wt, between 3%-6% wt/wt, between 3.5%-5.5% wt/wt, or between 4%-5% wt/wt of the lubricant 136.

The second component 104 may include a casing 128 having a proximal end and a distal end opposite the proximal end. The casing 128 may define a fluid path 130 therein. The fluid path 130 may be a generally cylindrical shape. The second component 104 may include a plug 132 disposed at the distal end thereof. The plug 132 may be a generally cylindrical member and may be fixed relative to the casing 128. The plug 132 may be disposed in the fluid path 130. The plug 132 may be a deformable member made of a rubber, silicone, or other deformable material. The plug 132 may include a septum 134 (not shown) extending therethrough. The septum 134 may be sized and shaped to receive a portion of the center post 112. The casing may further comprise an engagement arm 138 with a notch 140 extending therefrom. The engagement arm 138 may extend circumferentially around the casing 128 and may be spaced apart from the casing 128. The notch 140 may extend radially toward the casing 128. The engagement arm 138 and notch 140 may be sized and shaped to receive the collar 122 of the first component 102.

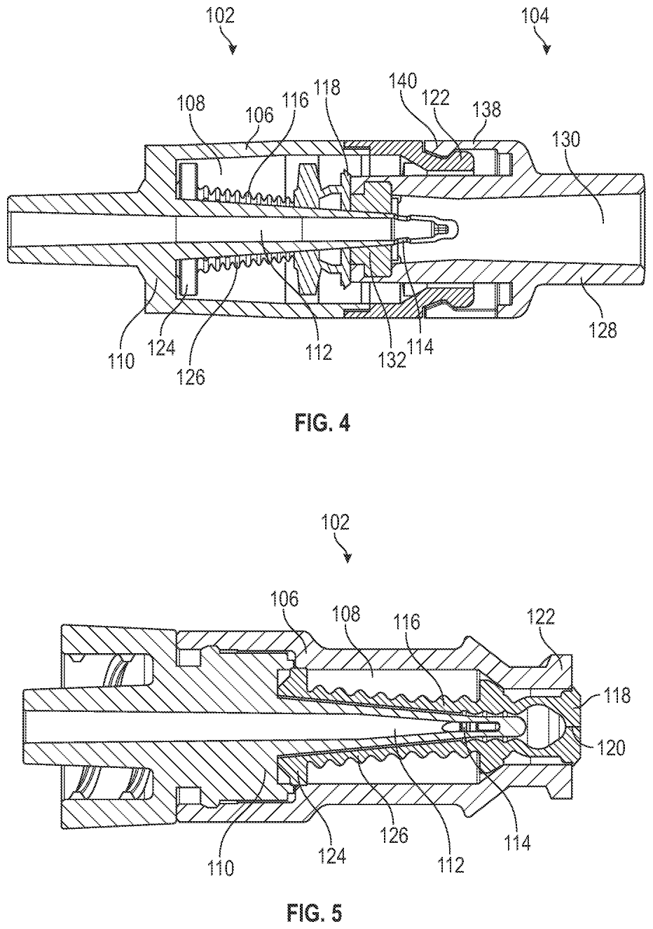

As shown in FIGS. 2 and 3, the first component 102 and the second component 104 may be spaced apart in a disconnected configuration. As shown in FIG. 4, the first component 102 and the second component 104 may be removably coupled in a connected configuration. The collar 122 of the first component 102 may pass by the notch 140 of the engagement arm 138 of the second arm in the connected configuration. The notch 140 may prevent the first component 102 from disconnecting from the second component 104 up to a threshold force. As discussed above, the notch 140 may allow the first component 102 to disconnect from the second component 104 to prevent a catheter from being unintentionally removed from a patient.

The first component 102 and the second component 104 may be in fluid communication in the connected configuration. The valve 116 may deform longitudinally in the distal direction to expose the center post 112 in the connected configuration. The distal end of the second component 104 may engage the base 118 to deform the valve 116. The center post 112 may be coated with the lubricant 136. The septum 134 may be coated with the lubricant when the center post 112 is disposed therethrough. The fluid medicament may travel through the fluid path 130, into the fluid inlet 114 and through the center post 112. In some embodiments, the fluid medicament may travel through the center post 112, out of the fluid inlet 114, and through the fluid path 130.

A method of reducing a presence of microbes in a needle free connector may be disclosed herein. The method may include providing a first component 102 comprising a valve 116 and a center post 112, providing a second component 104 comprising a plug 132, providing a lubricant 136 to an area between the center post 112 and the valve 116. The method may further include connecting the first component 102 and the second component 104 such that the plug 132 displaces the valve 116 and surrounds the center post 112, wherein the lubricant 136 is transferred to the plug 132 when the first component 102 is connected to the second component 104, and wherein the lubricant 136 comprises an antimicrobial component.

The antimicrobial component may comprises chlorohexidine gluconate. The concentration of the antimicrobial component may be between 4%-6% wt/wt of the lubricant. In some embodiments, the antimicrobial component may be at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, or at least 8%, of the lubricant 136. In some embodiments, the antimicrobial component may have a concentration between 1%-8% wt/wt, between 1.5%-7.5% wt/wt, between 2%-7% wt/wt, between 2.5%-6.5% wt/wt, between 3%-6% wt/wt, between 3.5%-5.5% wt/wt, or between 4%-5% wt/wt of the lubricant 136.

The first component 102 may further comprise a housing 106 having a proximal end and a distal end opposite the proximal end. The housing 106 may define an interior volume 108. The center post 112 may be disposed in the interior volume 108 extending proximally in the interior volume 108. The valve 116 may be disposed in the interior volume 108 at the proximal end of the first component 102 surrounding at least a portion of the center post 112.

The second component 104 may further comprise a casing 128 having a proximal end and a distal end opposite the proximal end. The casing 128 may define a fluid path 130 therein. The plug 132 may be disposed in the fluid path 130 at the distal end of the second component 104. The plug may comprise a septum 134 extending therethrough. The septum 134 may be sized and shaped to receive a portion of the center post 112. The septum 134 may be coated with the lubricant 136 when the center post 112 is disposed therethrough.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

What is claimed is:

1. A needle free connector comprising:
a first component comprising:
a housing having a proximal end and a distal end opposite the proximal end, the housing defining an interior volume;
a center post disposed in the interior volume extending proximally in the interior volume;
a valve disposed in the interior volume at the proximal end of the first component surrounding at least a portion of the center post; and
a lubricant disposed between the center post and the valve;
a second component comprising:
a casing having a proximal end and a distal end opposite the proximal end, the casing defining a fluid path therein; and
a plug disposed in the fluid path at the distal end of the second component;
wherein the lubricant comprises an antimicrobial component.

2. The needle free connector of claim 1, wherein the plug is a deformable member.

3. The needle free connector of claim 1, wherein the plug comprises a septum extending therethrough.

4. The needle free connector of claim 3, wherein the septum is sized and shaped to receive a portion of the center post.

5. The needle free connector of claim 4, wherein the septum is coated with the lubricant when the center post is disposed therethrough.

6. The needle free connector of claim 1, wherein the proximal end of the first component and the distal end of the second component are spaced apart in a disconnected configuration.

7. The needle free connector of claim 1, wherein the proximal end of the first component and the distal end of the second component are removably coupled in a connected configuration.

8. The needle free connector of claim 7, wherein the first component and the second component are in fluid communication in the connected configuration.

9. The needle free connector of claim 7, wherein the valve deforms longitudinally in the distal direction to expose the center post in the connected configuration.

10. The needle free connector of claim 1, wherein the center post is a rigid member.

11. The needle free connector of claim 1, wherein the antimicrobial component comprises chlorohexidine gluconate.

12. The needle free connector of claim 11, wherein a concentration of the antimicrobial component is between 4%-6% wt/wt of the lubricant.

13. A method of reducing a presence of microbes in a needle free connector, the method comprising:
providing a first component comprising a valve and a center post;
providing a second component comprising a plug;
providing a lubricant to an area between the center post and the valve;
connecting the first component and the second component such that the plug displaces the valve and surrounds the center post,
wherein the lubricant is transferred to the plug when the first component is connected to the second component, and
wherein the lubricant comprises an antimicrobial component.

14. The method of claim 13, wherein the first component further comprises:
a housing having a proximal end and a distal end opposite the proximal end, the housing defining an interior volume;
the center post disposed in the interior volume extending proximally in the interior volume; and
the valve disposed in the interior volume at the proximal end of the first component surrounding at least a portion of the center post.

15. The method of claim 13, wherein the second component further comprises:
a casing having a proximal end and a distal end opposite the proximal end, the casing defining a fluid path therein; and
the plug disposed in the fluid path at the distal end of the second component.

16. The method of claim 13, wherein the plug comprises a septum extending therethrough.

17. The method of claim 16, wherein the septum is sized and shaped to receive a portion of the center post.

18. The method of claim 17, wherein the septum is coated with the lubricant when the center post is disposed therethrough.

19. The method of claim 13, wherein the antimicrobial component comprises chlorohexidine gluconate.

20. The method of claim 19, wherein a concentration of the antimicrobial component is between 4%-6% wt/wt of the lubricant.
